(19) United States
(12) Patent Application Publication
(10) Pub. No.: US 2022/0211318 A1
(43) Pub. Date: Jul. 7, 2022

(54) MEDIAN POWER SPECTROGRAPHIC IMAGES AND DETECTION OF SEIZURE

(71) Applicant: CORNELL UNIVERSITY, Ithaca, NY (US)

(72) Inventors: Peter YAN, Ithaca, NY (US); Zachary GRINSPAN, Ithaca, NY (US)

(73) Assignee: CORNELL UNIVERSITY, Ithaca, NY (US)

(21) Appl. No.: 17/607,708

(22) PCT Filed: Apr. 29, 2020
(86) PCT No.: PCT/US20/30482

§ 371 Date: Oct. 29, 2021

Related U.S. Application Data
(60) Provisional application No. 62/839,853, filed on Apr. 29, 2019.

(51) Int. Cl. A61B 5/384; A61B 5/291; A61B 5/00

(52) U.S. Cl. CPC A61B 5/384; A61B 5/291; A61B 5/7257; A61B 5/7267; A61B 5/7405; A61B 5/746; A61B 5/4094

(57) ABSTRACT

Systems, methods and programs for processing EEG data for display and/or automatically detecting a seizure in a patient based on one or more spectrograms created from the EEG data. EEG data from a patient may be paired into channels based on electrode locations. Spectrograms are generated from EEG data from channels, respectively. The spectrograms of different channels are grouped and a median power spectrogram (MPS) is calculated for the group. The MPS may be used to automatically determine whether the patient had a seizure by applying a machined learned model (ML) model. The ML model is trained and tested using historical EEG data from a plurality of patients. The MPS or a relationship between a plurality of MPS of different groups may be displayed on a bedside monitor in real-time for viewing by a bedside clinician.

### CROSS-REFERENCE TO RELATED APPLICATIONS

This application claims the benefit of U.S. Provisional Application No. 62/839,853, filed on Apr. 29, 2019, the entire contents of which are incorporated herein by reference.

### FIELD OF THE DISCLOSURE

The present subject matter relates generally to modalities for processing electroencephalogram recordings of brain activity, displaying the results and automatically detecting a seizure.

### BACKGROUND

Non-convulsive seizures (NCS) affect 8-50% of critically ill patients, and are associated with nonconvulsive status epilepticus (NCSE), which has a high mortality rate of 17-51%. NCSE is regarded as persistent alteration in a patient's level of arousal and brain function, in the absence of any other observable physical manifestations. NCS can only be diagnosed in patients using an electroencephalogram (EEG), which is a clinical diagnostic test used to record electrical activity in the brain and detect seizures.

Access to rapid and accurate EEG seizure detection is generally very limited. This is particularly relevant in intensive care units (ICUs) where subtle and NCS are associated with high mortality, but difficult to diagnose. A continuous EEG (cEEG) is a clinical standard for diagnosing NCS. In a large (n=97) multicenter survey of tertiary care centers, 18% of ICU physicians would have increased cEEG duration had more resources been available, and at 17% of institutions there was no attending neurophysiologist EEG interpretation overnight. Furthermore, demand for cEEG is increasing, with 43% of institutions reporting an increase in cEEGs per month compared to the prior year. However, cEEG is very labor and time intensive to review. Even in large metropolitan hospitals with cEEG services, there are often delays between seizure onset and treatment. This is because cEEGs are typically reviewed intermittently rather than continuously monitored, and its review requires a subspecialist (neurophysiologist), who relays the EEG interpretation to the bedside clinician, who then decides on an intervention.

FIG. 1 illustrates an example of a known cEEG system. EEGs detect and record the electrical impulses through which neurons communicate by using electrodes/leads 5 attached to the scalp of a patient 2. The electrodes detect the electrical impulses in the brain and relay this signal via wires (leads) to an analog to digital converter (ADC 10). The signal is then digitalized and stored in a storage 20 on a server 15. It can then be subsequently retrieved by a computer 25 and displayed (usually as 18 different waveforms), to undergo visual analysis/review 30 by a clinical neurophysiologist 32. FIG. 4 illustrates an example of a display of the 18 different waveforms (channels, composed of electrode pairs) of sample clinical data. The first electrode in the channel label referenced to the second electrode (e.g. F3-C3 indicates that the F3 electrode is reference to the C3 electrode). The number of different EEG waveforms per screen may vary, typically between 15-20. As noted above, the waveforms may only be reviewed at certain times of day (and is not continuously monitored but rather intermediately). For example, the EEG waveforms may only be examiner 2 or 3 times a day.

Thus, a rapid and reliable way for the bedside clinician 36 to interpret the EEG would reduce treatment delays and improve patient outcomes, as rapid intervention is associated with increased success in treating NCS.

The major bottleneck to rapid EEG review is that the cEEG consists of 18 complex waveforms simultaneously displayed at 10-15 s epochs per screen, and the analysis is performed visually, screen by screen as shown in FIG. 4.

One potential solution is to process and visually summarize EEG data with quantitative EEG (qEEG) methods. These methods apply digital signal processing techniques to transform waveforms into spectrograms (spectrograms: images with frequency represented on the y-axis, time on the x-axis, and intensities for frequencies at a given time represented by a range of colors). This involves decomposing the complex waveforms into their individual frequency components, then generating a spectrogram that shows the change in power of these frequencies over time.

FIG. 7 depicts an example of the display with different process modes, e.g., visualization. These multi-modality qEEG visualizations may be used in clinical studies and sometimes in clinical practice. However, while these visualization are less laborious than the raw channels (waveforms as shown in FIG. 4), these qEEG visualizations are still complex and require extensive clinician training for appropriate interpretation.

One known qEEG method is the color density spectral array (CDSA) as shown in FIG. 6a. The CDSA, even when used in combination with multiple other qEEG methods (envelope trend, amplitude integrated EEG, asymmetry index, rhythmicity spectrogram) as part of a commercially available qEEG visualization tool (Persyst Inc., Prescott, Ariz.) clinically used by neurophysiologists, only achieved seizure identification sensitivity of 51-67%. Many qEEG displays are visually complex, employing multiple spectrograms or require simultaneous interpretation of very different visualizations as shown in FIG. 7. They therefore still require lengthy training (on the order of hours). Thus, more improvements in processing and displaying EEG information is needed, particularly to achieve rapid EEG review at the bedside for clinician immediately present rather than for the remote neurophysiologist.

After the clinical neurophysiologist 32 reviews the waveforms or the qEEG, the neurophysiologist 32 generates a report 35, based on the displayed waveforms and/or qEEG, which is subsequently sent to a bedside clinician 36 for intervention 40 as needed.

### SUMMARY

Accordingly, disclosed is a method comprising obtaining electroencephalogram (EEG) waveforms from a plurality of EEG channels, converting the received EEG waveforms into a spectrogram, respectively; grouping spectrograms corresponding to channels into a plurality of groups, for each group, aggregating the spectrograms into a median power spectrogram (MPS) calculating one or more relationships between the MPS from at least two groups; and displaying the one or more relationships on a bedside monitor. A channel comprises any pair-wise combination of EEG electrodes, respectively. The EEG electrodes may be paired according to a standard. Alternatively, in other aspects, the pairing may be application based. One electrode is designated as the active electrode and the other electrode as the reference. Each channel produces an EEG waveform. The spectrogram shows EEG spectral power as a function of frequency and time. At least two spectrograms are in each group. The electrodes are in contact with a scalp of a subject.

The channels may be grouped based on location of the electrodes on the scalp. For example, in an aspect of the disclosure, there may be four groups. The four groups may include anterior left and anterior right, posterior left and posterior right.

In an aspect of the disclosure, one of the relationships may be calculated by summing the MPS from at least two groups and displayed as a visualization. Additionally, the MPS from at least two other groups may be summed and displayed as another visualization.

In other aspects of the disclosure, one of the relationships is calculated by taking a difference between the MPS from at least two groups and displayed as visualizations.

In other aspects of the disclosure, both a sum and a difference of the MPS of different groups may be calculated and displayed as a visualization.

Each of the relationships may be separately displayed on a bedside monitor.

In an aspect of the disclosure, the MPS for the anterior left and the anterior right regions of the scalp may be summed and displayed as a visualization. Additionally, and/or alternatively, the MPS for the posterior left and the posterior right may be summed and displayed as a visualization. Further, additionally and/or alternatively, a difference between the MPS for the anterior left and the anterior right may be calculated and displayed as a visualization. Yet further, additionally and/or alternatively, a difference between the MPS for the posterior left and the posterior right may be calculated and displayed as a visualization.

In an aspect of the disclosure, the size and color of lines on the spectrograms are based on intensity and frequency. In an aspect of the disclosure, the MPS and the relationships between MPSs convey rhythmicity and intensity. For example, sloped harmonic bands indicate evolving rhythmicity.

In an aspect of the disclosure, the obtained EEG waveform, for each channel, may be scaled using the multi-taper spectral estimation method. The scaled EEG waveform may be converted into a spectrogram is based on a short time Fourier transform (STFT). In other aspects, the scaling may be omitted.

Seizures on the MPS are far easier to visually recognize compared to the standard EEG. This allows the bedside clinician to detect and intervene on seizures without relying on a neurophysiologist to interpret the EEG waveforms.

In an aspect of the disclosure, the method may further comprise automatically detecting a presence of a seizure.

In an aspect of the disclosure, the method may further comprise generating an alert when a seizure is automatically detected and transmitting the alert.

In an aspect of the disclosure, the method may further comprise, in response to receiving the alert, displaying the alert on the bedside monitor and/or generating a sound or transmitting the alert, by the bedside monitor in response to receiving the alert.

Also disclosed is a method comprising obtaining electroencephalogram (EEG) waveforms from a plurality of EEG channels, converting the obtained EEG waveform into an spectrogram, for each EEG waveform, grouping spectrograms corresponding to channels into a group, aggregating the spectrograms into a median power spectrogram (MPS) for the group; and determining whether the subject has a seizure using a model creates from a plurality of snapshot images of spectrograms from a plurality of patients and the MPS. A channel comprises any pair-wise combination of EEG electrodes, respectively. The electrodes are in contact with a scalp of a subject. The spectrogram shows EEG spectral power as a function of frequency and time.

In an aspect of the disclosure, the method may further comprise generating the model.

In an aspect of the disclosure, the model may be generated by obtaining a plurality of snapshot images of known seizures and a plurality of snapshot images of known non-seizures, dividing the plurality of snapshot images of known seizures and the plurality of snapshot images of known non-seizures into a training set of snapshot images and a testing set of snapshot images, classifying each snapshot image by applying an artificial neural network; for the training set of snapshot images, and testing the artificial neural network using the testing set of snapshot image.

In an aspect of the disclosure, the method may further comprise calculating an MPS for a plurality of groups; and calculating a relationship between the MPS from at least two groups.

In an aspect of the disclosure, the determination of the seizure may be based on the MPS and/or a relationship between MPSs for different groups. For example, the determination may include obtaining snapshot images from the MPS and/or snapshot images from the relationship between the MPSs using a moving window.

In an aspect of the disclosure, the subject or patient may be determined to have a seizure when a threshold number of consecutive snapshot images are classified as a seizure. For example, the threshold number may be 10.

In an aspect of the disclosure, snapshot images are obtained by a moving window with a set movement step.

In an aspect of the disclosure, historical EEG raw data from a database from a plurality of patients may be received. The historical EEG raw data may include EEG raw data from a plurality of patient determined to have a seizure and EEG raw data from a plurality of patients determined not to have a seizure. The raw data may be used to generate a MPS for each patient. For each MPS, snapshot images of the MPS are generated by using a moving window to generate a plurality of snapshots. Each snapshot is classified as a seizure image and non-seizure image.

In an aspect of the disclosure, the method may further comprise receiving a request from a client terminal to review the EEG waveforms and/or the MPS and in response to the request, transmitting the EEG waveforms and/or the MPS to the client terminal.

In an aspect of the disclosure, the artificial neural network may comprise a plurality of layers. The plurality of layers includes a plurality of layer sets. Each layer set having a different convolution operation. Each layer set has a convolution operation having X by X pixel convolution filters. X is the pixel size and is applied at Y-pixel steps. Y is the step size.

In an aspect of the disclosure, the number of X by X pixel convolution filters is different for each layer set.

Also disclosed is a server comprising a network interface, a storage and a processor.

The storage is configured to store digitized EEG signals received via the network interface. The EEG signals were obtained from electrodes in contact with a scalp of a subject. The EEG signals may be received from an acquisition device or directly from an analog to digital converter. The processor is configured to retrieve the EEG signals from the storage, group EEG signals into a plurality of EEG channels, where a channel comprises any pair-wise combination of EEG signals, respectively, convert the pair-wise combination of EEG signals of the channel into a spectrogram, for each channel, group spectrograms corresponding to channels into a plurality of groups, wherein at least two spectrograms are in each group, for each group, aggregate the spectrograms via a median power spectrogram (MPS), calculate one or more relationships between the MPS from at least two groups and transmit the MPS and/or the one or more relationships between the MPS from at least two groups to a bedside monitor. The spectrogram shows EEG spectral power as a function of frequency and time.

In an aspect of the disclosure, the processor may be further configured to automatically detect a seizure in a patient by analyzing the MPS and/or a relationship between the MPS from at least two groups.

In an aspect of the disclosure, the processor may be further configured to transmit an alert when a seizure is automatically detected.

In an aspect of the disclosure, the processor may be further configured to store the MPS and/or the one or more relationships between the MPS from at least two groups in the storage. As such, the processor may be further configured to receive via the network interface a request from a client terminal to view of the MPS and/or the one or more relationships between the MPS from at least two groups in the storage and in response to the receipt of the request, cause the transmission of the MPS and/or the one or more relationships between the MPS from at least two groups to the client terminal via the network interface.

Also disclosed is a server comprising a network interface, a storage and a processor.

The storage is configured to store digitized EEG signals received via the network interface. The EEG signals were obtained from electrodes in contact with a scalp of a patient. The EEG signals may be received from an acquisition device or directly from an analog to digital converter. The processor is configured to retrieve the EEG signals from the storage, group EEG signals into a plurality of EEG channels, where a channel comprises any pair-wise combination of EEG signals, respectively, convert the pair-wise combination of EEG signals of the channel into a spectrogram, for each channel, group spectrograms corresponding to channels into a group, aggregate the spectrograms via a median power spectrogram (MPS) for the group; and determine whether the subject has a seizure using a model creates from a plurality of snapshot images of spectrograms from a plurality of patients and the MPS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example of a known EEG system;

FIG. 2a illustrates an example of a EEG system in accordance with aspects of the disclosure;

FIG. 2b illustrates another example of a EEG system in accordance with aspects of the disclosure;

FIG. 2c illustrates an example of a method in accordance with aspects of the disclosure;

FIG. 3 illustrates an example of a seizure detection system in accordance with aspects of the disclosure, which also shows an example of a detected seizure with an alert/alarm;

FIG. 4 illustrates an example of the raw EEG data (waveforms) being displayed in a known EEG system;

FIG. 5 illustrates an example of a median power spectrogram in accordance with aspects of the disclosure;

FIG. 6a and FIG. 6b illustrate a comparison of a known processing and a median power spectrogram in accordance with aspects of the disclosure, where FIG. 6a shows a spectrogram from CDSA and FIG. 6b shows a median power spectrogram;

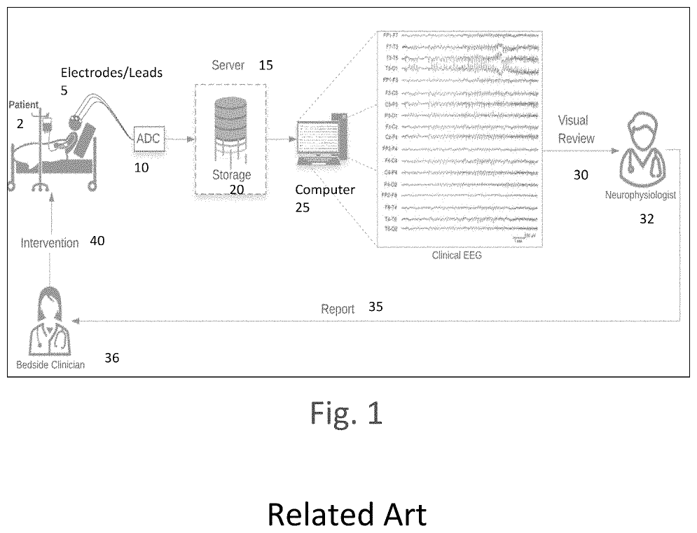

FIG. 1 illustrates an example qEEG display containing multiple different known qEEG visualizations;

FIG. 8d illustrates three main features examined in an median power spectrogram in accordance with aspects of the disclosure, with FIGS. 8a-8c inset showing examples of median power spectrograms;

FIGS. 9a-c illustrate different groupings of channels in accordance with aspects of the disclosure;

FIG. 10 illustrates an example of generating an median power spectrogram in accordance with aspects of the disclosure;

FIG. 11a illustrates an example of a waveform of a channel which is scaled and transformed into a spectrogram using multi-taper spectral estimation via a STFT in accordance with aspects of the disclosure;

FIG. 11b illustrates an example of a display depicting the sum median power spectrogram of different groups in accordance with aspects of the disclosure;

FIG. 11c illustrates an example of a display depicting differences of the median power spectrograms of different groups in accordance with aspects of the disclosure;

FIG. 12 illustrates a process for training and testing a machine learning model in accordance with aspect of the disclosure;

FIG. 13a and FIG. 13b illustrate an example of a sampling of a spectrogram of channels where a seizure is present in accordance with aspects of the disclosure;

FIG. 13c illustrates an example of a sampling of a spectrogram of channels where a seizure is not present in accordance with aspects of the disclosure;

FIG. 14 illustrates an example model in accordance with aspects of the disclosure;

FIG. 15a illustrates in a generic neural net configuration for the machine learning model used in example 2; FIG. 15b illustrates the different neural net configurations (Net 1-Net 4) based on the model illustrated in FIG. 15a;

FIG. 16a and FIG. 16b illustrate the training and cross-validation results of the neural network models in example 2;

FIG. 17 illustrates the performance of the neural network models in detecting seizures on spectrograms of EEGs from CHB-MIT; and

FIG. 18 illustrates the performance of the neural network models in detecting seizures on spectrograms of EEG from WCMC.

### DETAILED DESCRIPTION

The technology disclosed herein is directed to a novel qEEG and spectrogram visualization method comprised of using multi-taper spectral estimation and aggregating spectral power over regions (groups of channels of the scalp using medians (MPS)). Seizures on MPS are easily to visually recognize.

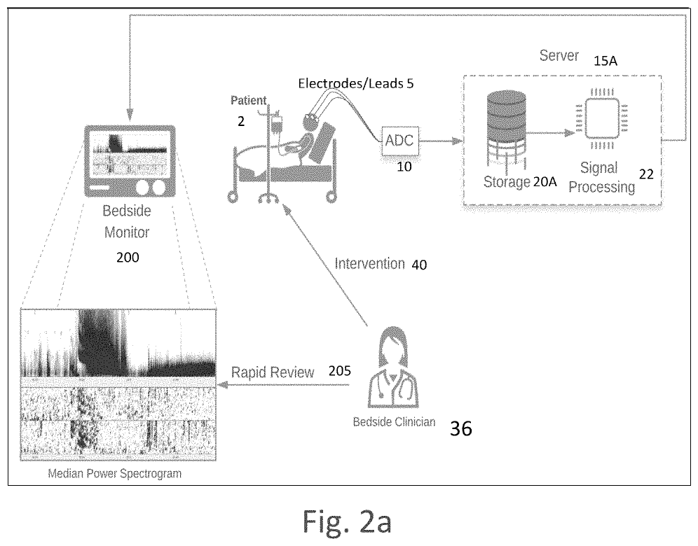

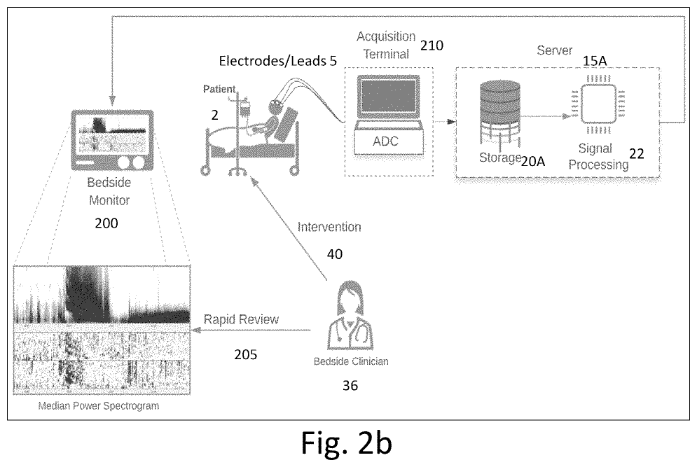

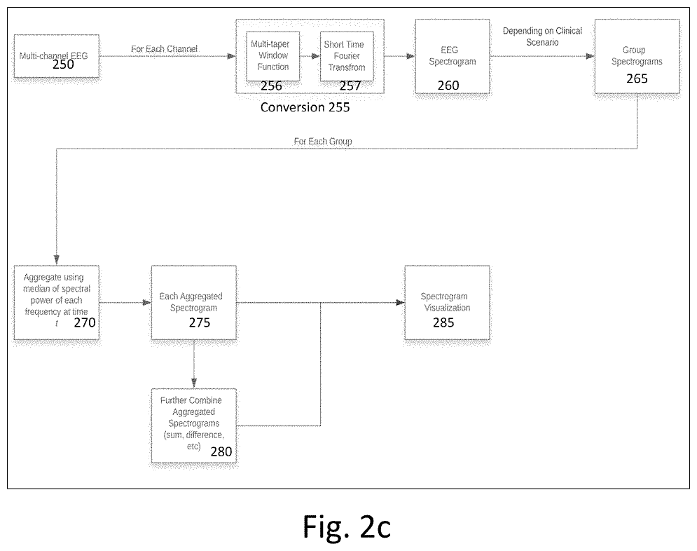

FIG. 2a illustrates an example of the novel qEEG system in accordance with aspects of the disclosure. The system comprises a plurality of electrodes/leads 5. The EEG electrodes/leads 5 may be any number, paired into channels in any configuration. The pairing may be according to an industry standard. In other aspects of the disclosure, the pairing of electrodes may be customized as desired. The pairing creates the EEG waveform, e.g., difference in voltages between the electrodes. The signals from the EEG electrodes/leads 5 are converted into a digital signal for processing by an ADC 10. The ADC is set with a predetermined sampling rate. The predetermined sampling rate is based on the frequency band of interest, including harmonics. The digital signals are sent to a server 15A via a network.

The server 15A includes storage 20A and signal processing 22. For example, the server 15A may comprise a processor such as, but not limited to a CPU. The CPU may be configured to execute one or more programs stored in a computer readable storage device such as the storage 20A. For example, the CPU may be configured to execute a program causing the CPU to perform the functions described herein such as generating median power spectrograms(s) (MPS) and generating relationships between the MPS of groups for display. In other aspects, the processing may be executed in a GPA or other hardware, such as but not limited to an ASIC or FPGA.

The storage 20A may be, but not limited to, RAM, ROM and persistent storage. The memory 20A is any piece of hardware that is capable of storing information, such as, for example without limitation, data, programs, instructions, program code, and/or other suitable information, either on a temporary basis and/or a permanent basis. In some aspects, the disclosure, the storage 20A stores the received digital signals from the ADC for processing and display on a client terminal (not shown). A client terminal may assess the server 15A and view the EEG waveforms of the channels. This may be done to confirm the visualizations displayed on the bedside monitor 200. In some aspects of the disclosure, the server 15A may be assessed by the client terminal via the Internet and a secured login. In other aspects of the disclosure, the client terminal may also request to view of the MPS and/or the one or more relationships.

The server 15A may also comprise a wireless communication interface (not shown in FIG. 2a). The wireless communication interface may be configured to wirelessly communicate with the bedside monitor 200 and the ADC 10. In other aspects of the disclosure, the server 15A may comprise a wired communication interface (also not shown in FIG. 2a) which is connected to a local area network (LAN) or other network for communication with a bedside monitor 200 and the ADC 10.

In other aspects of the disclosure as shown in FIG. 2b, the system may further comprise an acquisition terminal 210 in the patient's room. The acquisition terminal 210 may include the ADC. In other aspects, the ADC 10 may be separate and connected to the acquisition terminal 210. The acquisition terminal 210 acquires the digitized signals from the ADC 10. This acquisition terminal 210 may confirm the data (temporarily storing then checking for missing bits) and relay the digitized signals to the server 15A via a network. As such, the acquisition terminal 210 may also include a network interface. For example, the acquisition terminal 210 may comprise a wired interface or wireless interface.

The bedside monitor 200 comprises a display. The display is a color display. The size the display may be limited which is another advantage of using the MPS instead of the known qEEGs. The display is capable of displaying one or more relationships between the MPSs of groups for rapid bedside seizure detection via rapid spectrogram review 205. An MPS for a group may also be displayed in some aspects of the disclosure. The bedside monitor 200 also comprises a network interface. Similar to the server 15A, the network interface may be a wired or wireless interface. The bedside monitor 200 may receive the one or more relationships from the server 15A. In other aspects, the bedside monitor 200 may also receive the MPS of one or more groups for display.

The bedside monitor 200 may display a plurality of minutes to hours of the MPS relationships (or the MPS) which can be viewed at once by the bedside clinician 36, with seizures appearing visually distinct. The system enables the bedside clinician 36 to rapidly review 205 a spectrogram (without a significant amount of training, and intervene (intervention 40) if seizures are detected, without waiting for the neurophysiologist's interpretation. Advantageously, in accordance with aspects of the disclosure, intervention 40 may be applied quicker than with the known systems. This is at least because (1) the bedside monitor displays the relationships of the MPS, which is easier to interpret as the MPS is colored image representing frequency-intensities with leads to more accurate and efficient detection, (2) less processing modes are displayed, reducing confusion, (3) eliminates a need to scroll through channels of data or change screens and (4) eliminates the need to wait for the neurophysiologist's interpretation.

While FIGS. 2a and 2b show only one bedside monitor 200, the server 15A may communicate with a plurality of bedside monitors 200 and respective ADC 10 and/or acquisition terminals 210 and cause the display of the MPS and/or one or more relationships of the MPSs on the plurality of bedside monitors 200 for different patients 2, respectively.

FIG. 2c illustrates a flow diagram of generating the MPS for display (displaying) and determining the relationships. FIG. 10 also depicts a process of aggregating channels into the MPS. In accordance with aspects of the disclosure, the brain activity of a patient 2 is recorded with electrodes/leads 5 and the recordings are processed and displayed in order to determine a likelihood that the patient 2 is having a seizure. Any device suitable for recording EEG data and configured to collect EEG data via the scalp of the patient 2 via electrodes may be used. The electrodes are placed on the scalp of the patient 2. The electrode 2 may be placed in preset locations according to a standard.

As noted above, the electrodes are paired to create an EEG waveform, one electrode is an active electrode and the other electrode in the pair is a reference. The EEG waveform is the difference in the voltage between the active electrode and reference electrode (as described herein as a channel). FIG. 11A shows an example of an EEG channel from an electrode-pair (left side). The signals from the electrodes/leads 5 are digitized by ADC 10, transmitted to the server 15A and stored in storage 20A (FIG. 2c, 250).

The signals are subject to signal processing 22 in the server 15A by a processor, e.g., the CPU. The CPU converts the waveforms from the time domain into a frequency domain (FIG. 2c, conversion 255).

In some aspects of the disclosure, the CPU converts the EEG waveforms (channels) to spectrograms by executing a short time Fourier transform (STFT) (FIG. 2c, 257). A STFT is a moving window (over time) that calculates a discrete Fourier transform (DFT). The STFT window can be of any width and advance by any time increment, so long as the window width is greater than the time increment. In an aspect of the disclosure, a window width of 2 s and a time increment of is may be used and produces a spectrogram of sufficient temporal resolution for visualizing evolving seizure activity. However, the width and time increment is not limited to 2 s and 1 s, respectively.

In an aspect of the disclosure, prior to the STFT, the waveform may be scaled via any number of tapers (scaling functions) (FIG. 2c, 256), to set the level of compromise between frequency resolution (generally desirable for a high resolution spectrogram) and spectral leak (generally undesirable as the spectral power representation in the spectrogram becomes less accurate); frequency resolution and spectral leakage are inversely correlated and a fundamental property of all Fourier transforms.

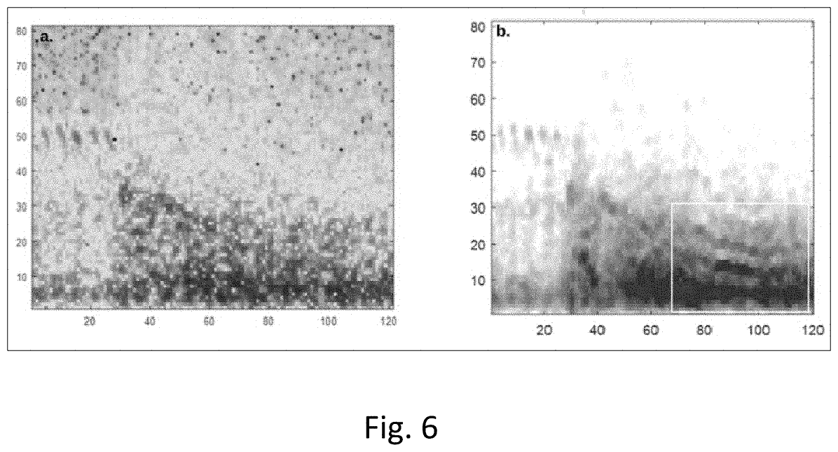

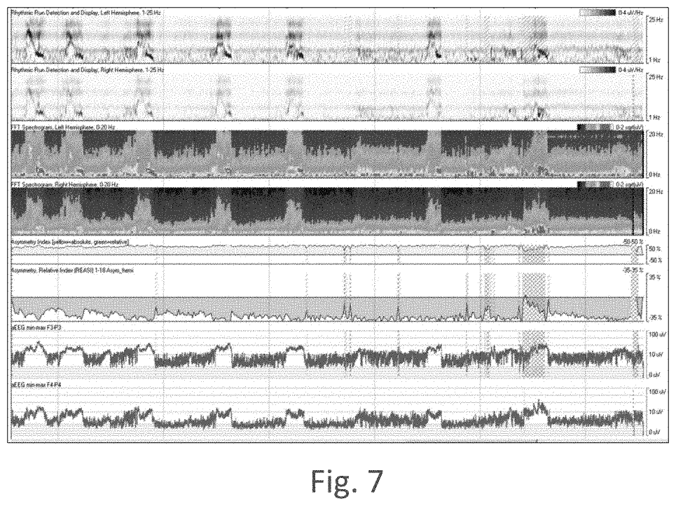

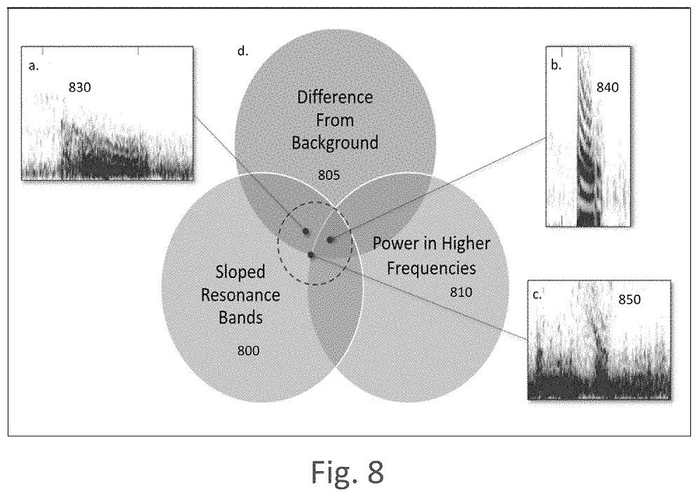

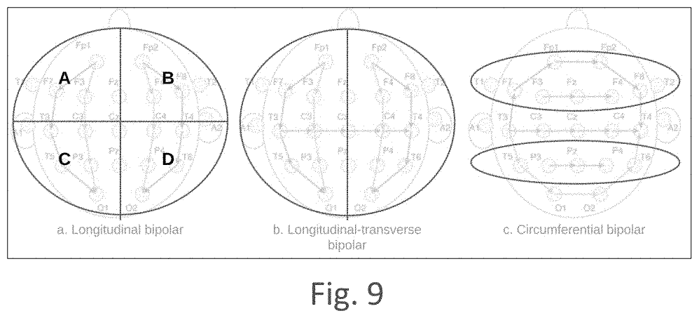

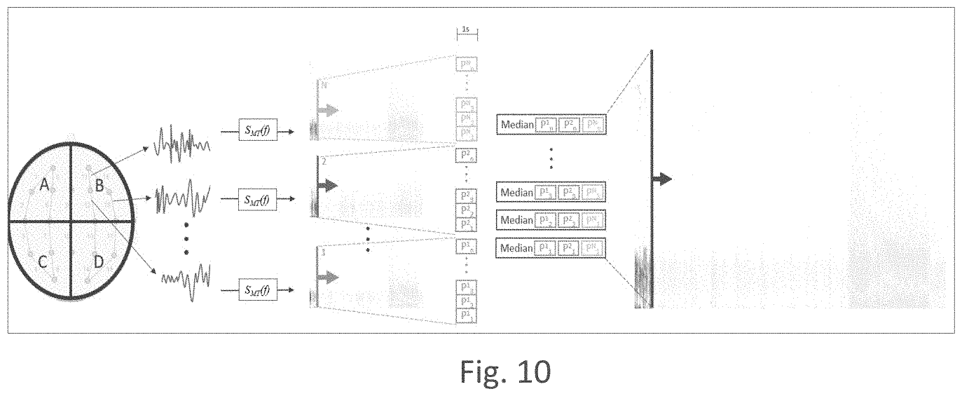

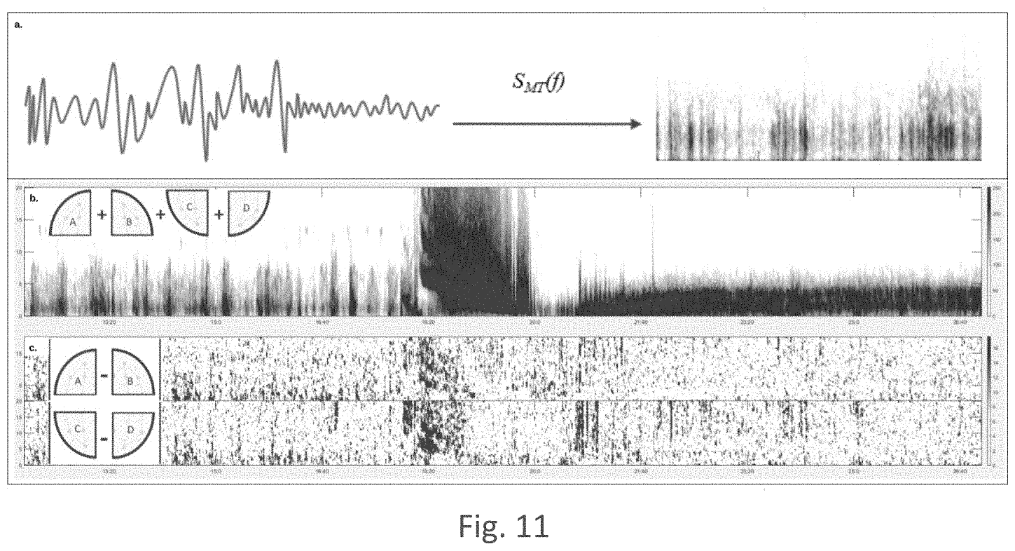

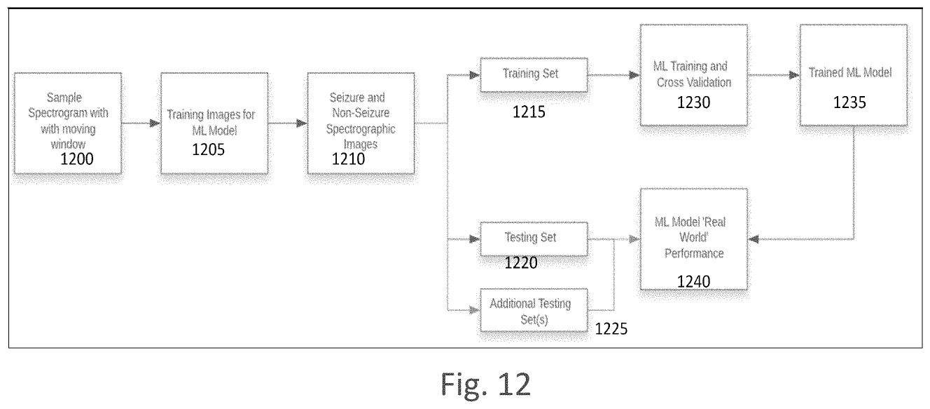

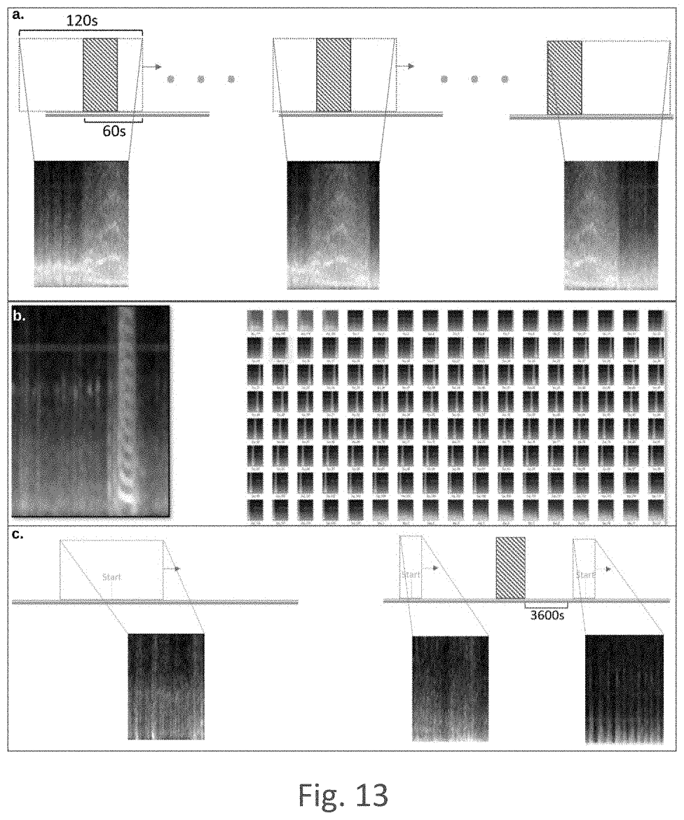

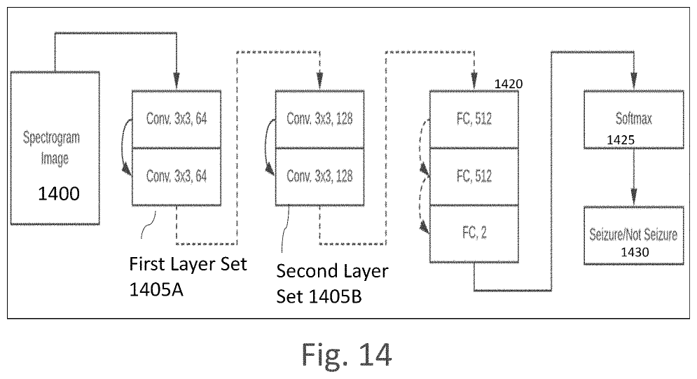

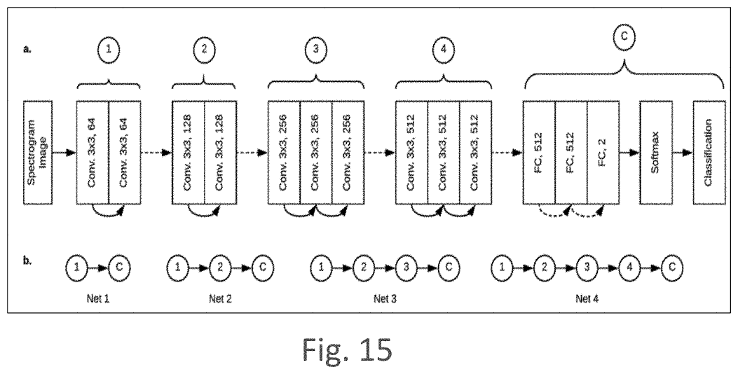

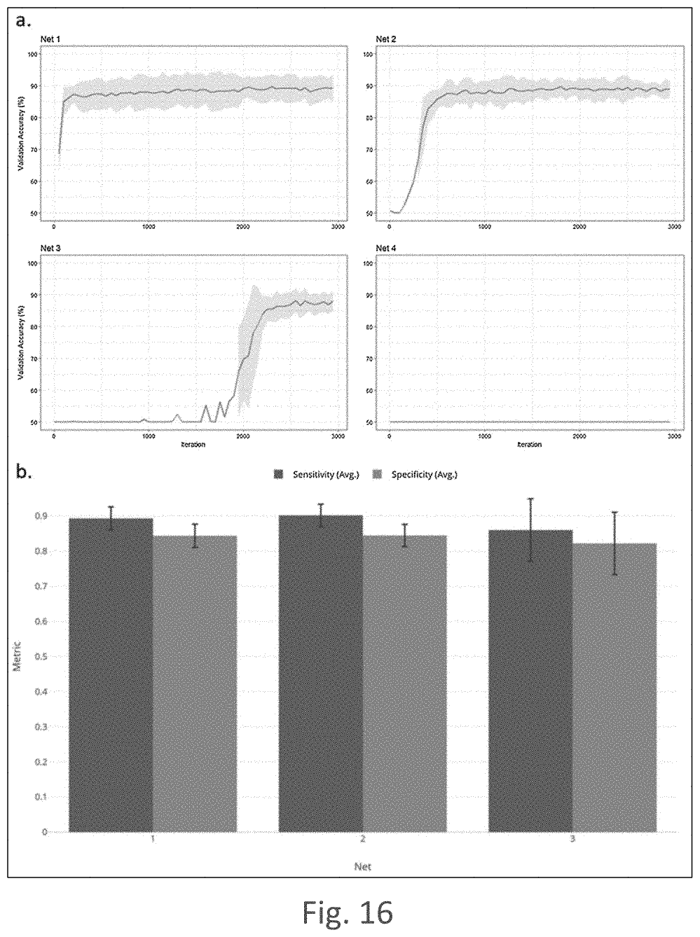

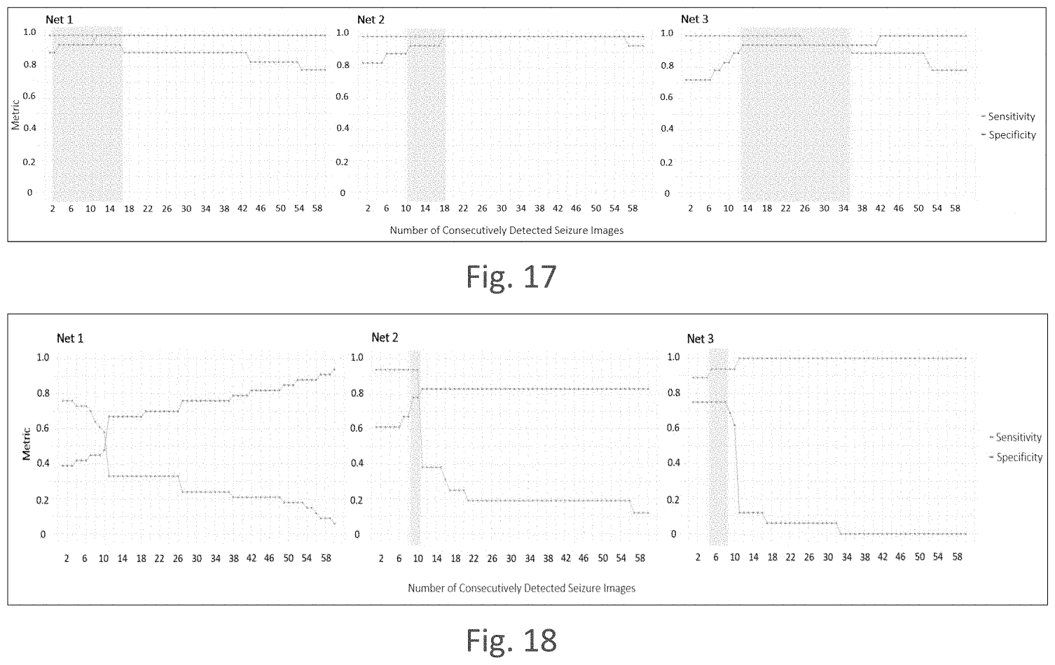

The multi-taper spectral estimation is a method that maximizes frequency resolution while minimizing the spectral leakage that occurs when transforming time domain data (e.g., waveforms) to frequency domain representations (e.g., spectrograms). Multi-taper spectral estimation may be applied to statistically determine optimal tapers that maximize frequency resolution while minimizing spectral leakage. In an aspect of the disclosure, to optimize frequency resolution, a multi-taper spectral estimation with parameters: K=2, WT=2, DFT size 4096, yields a frequency resolution of 2 Hz may be used. This offers sufficient resolution for visualizing seizure specific features on the MPS such as shown in FIG. 6b, while allowing for near real time spectrogram generation by the server 15A.

FIG. 11a shows the output of the STFT/multi-taper spectral estimation for one channel (spectra) or spectrogram (FIG. 2c, 260). The STFT/multi-taper spectral estimation results in n frequency bins for each channel. FIG. 10 illustrates a representation of the power for different channels. For each 1 through Nth spectrogram, the power is denoted as P¹, P², . . . P^{N} in FIG. 10 (where “N” is the number of channels in the group). For different channels, the power corresponding to the 1st through N-th channel is shown in FIG. 10 using different colors. For example, power for the first channel is shown in orange, power for the second channel is shown in green and power for the Nth channel is shown in yellow). The power corresponding to the 1st through n-th frequency bin for the Nth spectrogram is denoted as P^{N}₁, P^{N}₂, . . . P^{N}ₙ.

In accordance with aspects of the disclosure, different channels are grouped into different groups (FIG. 2c, group spectrograms 265). The channels may be distributed evenly or unevenly among a plurality of groups. In another aspect, the groups may be of varying sizes and locations (e.g. divided by quadrants on the scalp) along the subject's scalp. In another aspect, EEG from the various grouped electrodes are analyzed together or independently.

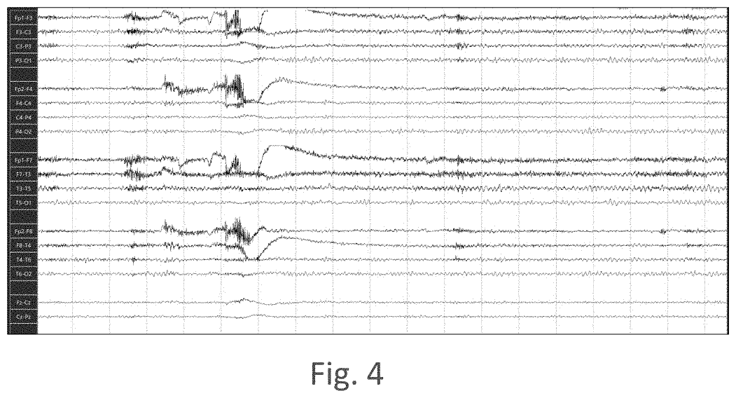

FIGS. 2a-9c depicted different groupings of the channels. The blue circles represent example groupings. In FIG. 9A, the channels are grouped into four groups: A, B, C, and D. The grouping includes anterior left, the anterior right, posterior left and posterior right (a longitudinal bipolar arrangement). This grouping is similar to the grouping shown in FIG. 10. In the example depicted in FIG. 10, Group A: Fp1-F7, F7-T3, Fp1-F3, F3-C3, Fz-Cz, Group B: Fz-Cz, Fp2-F4, F4-C4, Fp2-F8, F8-T4, Group C: T3-T5, T5-O1, C3-P3, P3-O1, Cz-Pz, and Group D: Cz-Pz, C4-P4, P4-O2, T4-T6, T6-O2.

In FIG. 9b, the channels are groups into two groups: left and right hemispheres (longitudinal-transverse bipolar). FIG. 9c shows another grouping of channels in two groups (circumferential bipolar). The individual arrows in FIGS. 9a-9c and 10 represent the pairs of electrodes which form a channel.

EEG electrodes can be paired in a multitude of ways creating waveforms that may better represent the underlying brain activity, depending on the clinical scenario. Pairing of electrodes may occur at 250 in FIG. 2c.

FIG. 10 shows a representation of the power for group A.

Spectrograms from each channel can be aggregated based on their grouping and visualized. In accordance with aspects of the disclosure, the spectrograms from channels in a group are aggregated by the median power (FIG. 2c, 270).

The median is less sensitive to signal outliers compared to the mean. For EEG data, this may mitigate spurious signals from a malfunctioning electrodes or muscle activity. Combining the multi-taper spectral estimation and median of spectral power results in a median power spectrogram (MPS) that is easy to interpret and can be quickly taught to clinicians.

The reason for the MPS's easy interpretability is that it generates images with patterns that are visually distinct and specific for seizures. The inventors have determined that seizures have three distinctive features in the MPS that enable the use of the MPS (and relationships therein) to detect seizures. The seizure may cause in the MPS a sloped resonant band 800, difference from background 805 and power in high frequencies 810. Examples of the three different MPSs are shown inset in FIG. 8d (FIGS. 8a-8c). In FIG. 8b, the MPS 840 exhibits all three distinct features being prominent. In FIGS. 8a and 8c, one or more features are less prominent. In FIG. 8a, the MPS 830 has some sloped resonant bands 800 and difference from background 805. The MPS 850 in FIG. 8c has some power in high frequencies 810, some difference from background 805 and some sloped resonant band 800. Depending on the seizure, one or more of the features may be more prominent. Advantageously, the MPS is able to display these features distinctly.

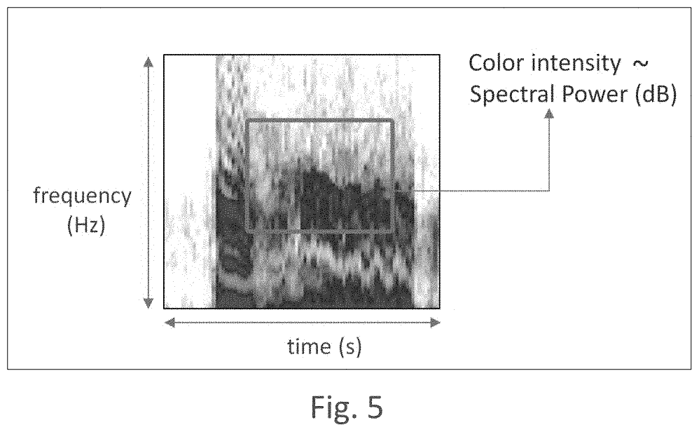

In an aspect of the disclosure, the CPU determines the median of the power in each frequency across the channels of the group, e.g., 1 through nth frequency bin. The medium power of each frequency may be determined per second. This aggregation creates the median power spectrograms (FIG. 2c, 275). For example, for the first frequency, e.g., P₁, the CPU determines the median power across the N channels in the group, e.g., Group A (at a time T). This is shown in FIG. 10 as Median P¹₁, P²₁, . . . P^{N}₁. This is simultaneously done for each frequency bin. FIG. 5 illustrates an example MPS. As with all spectrograms, starting from the lower left corner, frequency increases with the vertical dimension of the image and time increases with the horizontal dimension. The spectral power of a given frequency at a given time is represented as color, with usually the more intense color (red—in this spectrogram) representing higher power. The spectral power in FIG. 5 is shown in dBs.

The MPS may be generated in near time, transmitted from the server 15A to the bedside monitor 200 and the displayed, showing the MPS and/or the one or more relationships (the real-time display is shown in FIG. 10 by the arrow showing the MPS as it is created (right direction represents time)).

The MPS is determined for each Group, e.g., in FIG. 10, “A-D”, creating a visualization for each group. For the example shown in FIG. 10, there would be four different MPS, one per group. In other examples, such as FIGS. 9b and 9c, there may be two MPS.

FIGS. 6a and 6b show a comparison of spectrogram from color density spectral array (CDSA) versus the MPS for an example image of a seizure in accordance with aspects of the disclosure. For this seizure, the CDSA is unable to resolve the evolving harmonic bands, whereas these bands are visually distinctive in the MPS in accordance with aspects of the disclosure, which is shown in a white box in FIG. 6b.

Additionally, in an aspect of the disclosure, relationships between MPS of different groups may be determined (FIG. 2c, 280) and displayed as visualization (FIG. 2c, 285). The relationships may be summed or a difference between different MPS of the different groups. This produces different condensed visualizations.

In some aspects, the MPS of any two groups may be added together to create a new visualization. In other aspects, the sum of the MPS of any two groups may also be added together also creating a new visualization. FIG. 11b illustrates an example of such a relationship. In FIG. 11b, the MPS from group A and B (two quadrants) were added and the sum of the MPS from Group C and D (two quadrants) were added and both sums were subsequently added together. The time is labeled on the x-axis. The frequency is labeled on the left of the figure (y-axis) and the intensity scale is shown on the right. Advantageously, this summed MPS provides a better visualization of the EEG activity over the entire scalp.

In some aspects, the MPS of any group may be subtracted from the MPS of another group. FIG. 11c illustrates an example of the MPS of different groups being subtracted. In FIG. 11c, the MPS of Group B is subtracted from the MPS of Group A (top of FIG. 11c) and the MPS of Group D is subtracted from the MPS of Group C (bottom of FIG. 11c). In this example, the differences taken across the anterior and posterior scalp quadrants allow for better visualization of focal activity within the anterior or posterior scalp. While FIG. 11c shows two differences, only one difference may be taken, depending on a region of interest. Additionally, the groups in the specific views may be changed. For example, the MPS of Group D may be subtracted from the MPS of Group B. Further, the MPS of Group C may be subtracted from the MPS of Group A.

Some seizures can be seen in both a summed MPS (S-MPS) and a difference MPS (D-MPS). For example, as a focal seizure propagates across the head, the spectral power becomes increasingly prominent in an S-MPS.

The server 15A transmits the MPS (FIG. 2c, 275) or the relationships (aggregations of MPSs) (FIG. 2c, 280) to the bedside monitor 200 for display (FIG. 2c, 285).

In other aspects, a system and method for automatically detecting a seizure is disclosed.

Because qEEG methods transform EEG waveforms into spectrograms such as described herein, which are effectively colored images, these images can be used to train machine learning models. These trained models can then automatically detect presence of seizures on the spectrograms and alert the bedside clinician. In particular, as described above, the MPS with seizures may have distinct features that can be used as salient training images for machine learning models that can be trained to recognize seizures on the MPS, automating the seizure detection process.

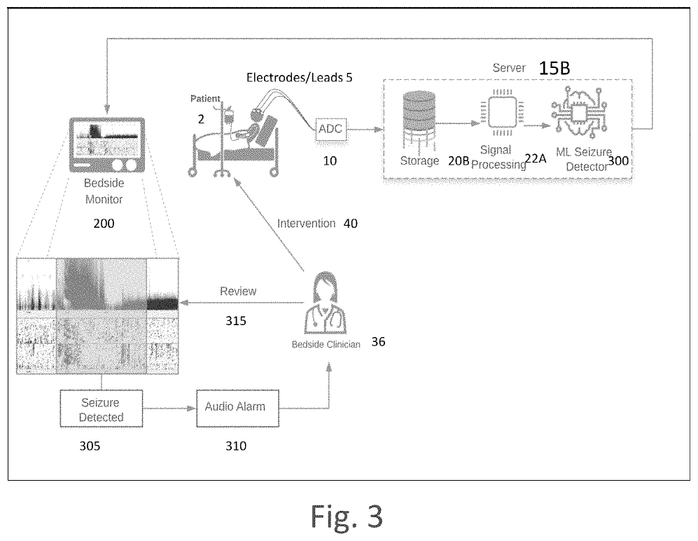

FIG. 3 illustrates a system for automatically detecting a presence of a seizure in a patient 2 and generating an alert in accordance with aspects of the disclosure. Many of the components of the system are the same as described above and will not be described again. Similar to FIG. 2a, the server 15B receives a digitized output from the ADC 10 (or relayed from the acquisition terminal 210 as shown in FIG. 2b). The acquisition terminal 210 is not shown in FIG. 3. The server 15B may comprise a storage 20B, signal processing 22A and a machine learning (ML) seizure detector 300. A processor, such as, but not limited to a CPU in the server 15B may perform the signal processing 22A and the ML seizure detector 300. In an aspect of the disclosure, a single CPU may execute both. In other aspects of the disclosure, different CPUs may collectively execute the signal processing 22A and the ML seizure detector 300. The signal processing 22A may be the same as described above to generate a MPS (spectrograms). In other aspects, other types of spectrograms may be used such as average spectrograms. Like in FIG. 2c, the visualizations, such as MPS (FIG. 2c, 275) and/or one or more relationships (FIG. 2c, 280) may be transmitted to the bedside monitor 200 for confirmation of the automated seizure detection (similar to FIG. 2c, 285).

In some aspects, the storage 20B stores the received digital signals from the ADC (or acquisition device 210) for processing and display on a client terminal (not shown). A client terminal may assess the server 15B and view the EEG waveforms of the channels. This may be done to confirm the detection. In some aspects of the disclosure, the server 15B may be assessed by the client terminal via the Internet and a secured login. In other aspects of the disclosure, the client terminal may also request to view of the MPS and/or the one or more relationships.

The bedside monitor 200 may also comprise a light emitter or speaker. The light emitter or speaker may generate an alert (such as a visual or audio alarm 310) when a seizure is automatically detected (305). The spectrograms, such as the S-MPS (and/or MPS) and/or other relationships showing the detected seizure area may also be displayed on the bedside monitor 200 for review and confirmation 315. In some aspects of the disclosure, a window may be superposed on the display area over the frames which were classified as a seizure 305. The clinician 36 can then rapidly review 315 the spectrogram on the bedside monitor 200 to confirm the automated detection and provide intervention 40 as needed. Advantageously, the automatic detection and alarm/alert eliminates a need for continuous monitoring the EEG channels by the clinician 36.

FIG. 12 illustrates a process for training and testing the server 15B (machine learning model) to automatically detect a seizure.

Because spectrograms are digital images, a myriad of machine learning models can be used. The selected machine learning model is trained on consecutive snapshots of spectrograms that contain seizures as well as those that do not contain seizures. The model thus learns to distinguish spectrograms with seizures from those without.

EEG raw data (EEG data) is obtained from a data repository for a plurality of patients, patients that were determined by board certified neurophysiologists to have a seizure and patients determined not to have seizures. The EEG raw data may be obtained from one or more hospital records. Thus, in an aspect of the disclosure, the server 15B may also have a network interface to communicate with different hospital systems such as clinical databases. The server 15B also obtains a predetermined classification of the EEG raw data. This EEG raw data may be classified based on a system described in FIG. 1, e.g., a neurophysiologist, looking at the EEG raw data. The server 15 generates one or more spectrograms for each patient. For example, the spectrograms may be generates in a manner described above. In some aspects, the CPU may generate a relationship for the MPS, such as an S-MPS for the scalp.

In an aspect of the disclosure, as the spectrogram is generated in near real time, a snapshot (frame) of the spectrogram is sent (from the signal processing 22A) to a machine learning model (ML seizure detector 300) to detect if a seizure is present (1200).

In an aspect of the disclosure, spectrograms containing seizures are sampled with snapshots that capture a predetermined window of time, where the seizure is occurring in the middle of the window. For example, the predetermined window of time may be 120 s.

Snapshots are then taken at predetermined times intervals as the seizure advances across the window. For example, the interval may be 1 s. This results in a number I of snapshots (e.g., 60 snapshots per seizure). The snapshots are labeled for confirmation and verification of the training.

FIGS. 13a and 13b show snapshots of a sample spectrogram containing a seizure. The seizure within the spectrogram is schematically depicted as a shaded rectangle. In the example, the width of the snapshot window is 120 s in duration, with the initial snapshot taken with the onset of seizure in the middle of the window. The window then advances by 1 s increments, with a snapshot taken at each increment. The window takes a snapshot of the region of the spectrogram containing the seizure, and then takes consecutive snapshots across the region. This continues until the seizure's onset reaches the left most edge of the window. FIG. 13b shows a plurality of snapshots which shows the seizure moving with the snapshots from the right to the left as time passes. FIG. 13b shows 136 snapshots where one of the snapshots is enlarged. The snapshots of seizure movement in FIG. 13b mimic its movement on a bedside monitor 200 showing a visualization over time.

The spectrograms without seizures are sampled at random non-overlapping positions in a similar manner as the seizure snapshots above. In a spectrogram without seizures, a random location (after 60 s and before the last 60 seconds of the spectrogram) is selected as the start point. The snapshots begin with the starting pointing in the middle of a 120 s window, with advances by 1 s, as shown in FIG. 13c-left. Additionally, non-seizure parts of spectrograms with seizures are also sampled, to provide more diversity in these samples for training later. When sampling non-seizure regions in spectrograms with seizures, as shown in FIG. 13c-right, the random starting points are constrained to at least 60 s prior to seizure onset (so that the window does not eventually overlap with the seizure) and 3600 s after the seizure (to avoid sampling residual effects of the seizure).

The above generates the images for training and testing (FIG. 12, 1205).

These snapshots are then used to train 1230 and test 1240 a layered convolutional neural network (CNN) (an artificial neural network, a specific type of machine learning model adept at image recognition). The training is supervised learning where the machine learning model is presented labeled snapshots (i.e. seizure or no seizure) as the ground truth and the model then proceeds to learn (i.e. adjust its internal parameters) to correctly distinguish between the seizure and non-seizure snap shots. The labeling is used to determine which snapshot method is used, e.g., snapshot method for seizure (FIGS. 13a and 13b) verses non-seizure (FIG. 13c)

Using the predetermined classification (including start and end time), the server 15B, e.g., CPU divides the snapshot images into two groups, seizures and non-seizures 1210.

In other aspects of the snapshot images are manually divided by a client or operator by visual inspection.

A certain number of snapshot images from the seizure group and a certain number of snapshot images from the non-seizure group are selected for training (FIG. 12, 1215). In an aspect of the disclosure, the selection may be random. Remaining snapshot images from each group may be used for testing and confirmation of the model (FIG. 12, 1220).

FIG. 14 illustrates an example of a CNN in accordance with aspects of the disclosure. The CNN is composed of sequential layers of convolution and sub-sampling operations.

In some aspects of the disclosure, the CNN is of a VGG-net configuration. The CNN comprises a plurality of layer sets 1405. In FIG. 14, two convolution layer sets 1405A and 1405B are shown. However, in other aspects of the disclosure, a different number of layer sets may be used. Each layer set 1405 has a plurality of layers. As depicted, each layer set 1405 has the same number of layers, however, in other aspects, the number of layers may be different. For example, one layer set may have two layers and other layer set may be three layers. The resolution of the layers in the blocks may be different. For example, in first layer set 1405A, the convolution layers may have a first resolution, such as 64 (as depicted in FIG. 14). The layers in a second layer set 1405B may have a second resolution such as 128 (as depicted in FIG. 14).

Each convolution layer has a M×M pixel size. For example, as depicted in FIG. 14, each layer has a 3×3 pixel size.

The convolution layer sets 1405 are connected in series. The output of each convolution layer set is a set of ‘higher-level’ feature representations that describe the input (feature representations from the previous convolution layer set), which is originally derived from the input image (snapshot). This new representation of the input image (snapshot) can then be further processed by additional convolution layer sets, until the final ‘optimal’ feature representation of the input image is learned in the other layers (FC units 1420 and softmax 1425).

The result from each layer is then passed through rectified linear units to the next convolution layer (solid curved arrows). Results are then passed to the next layer set via 2×2 max pooling (dashed right-angle arrows). Finally, features from the convolution layers (are passed to the FC units 1420.

The CNN comprises a plurality of full connected (FC) units 1420 of artificial neuron layers. Between each layer there is dropout (dashed curved arrows). The FC units 1420 may also have a different resolution. For example, in FIG. 14, two of the FC units have a resolution of 512 and the other has 2. The output of the FC units 1420 is passed to a softmax 1425, which leads to a classification 1430 of the spectrogram image (snapshot) 1400 (no seizure or seizure). Softmax 1425 is a well known function and will not be described herein in detail.

The spectrogram image(s) 1400 in FIG. 14 is/are the snapshots described above. All snapshots from the selected snapshots for both non-seizure and seizure are input in the CNN for training and cross-validation.

The training is designed to determine the optimal weight for convergence, e.g., extract the optimal features for describing a spectrographic seizure in the snapshots(s). Prior to testing, cross-validation is used. During cross-validation, the training data is divided into parts (typically 5 or 10), then the machine learning model is trained on all but one randomly selected part and then the model's performance on the remaining selected part is obtained. This process is repeated for a pre-specified number of times, and the model's performance is recorded each time then aggregated. The purpose of cross-validation is to determine the stability of the model (e.g., consistency of convergence in artificial neural nets) and provide a general idea of how the model will perform on the official testing phase later (e.g., models that perform poorly during cross-validation are often eliminated and not considered worth testing). Once the optimal weights are determined and cross-validated, the trained model is output for testing 1235

The trained model 1235 may subsequently be tested (FIG. 12, 1240) using the testing set 1220 as described above and/or with additional testing snapshot images 1225. The additional testing set 1225 are converted into a spectrogram such as using the method described above. The spectrogram data is sampled and consecutive snapshots are generated as described above. In an aspect of the disclosure, while the CNN is being trained, the CPU in the server 15B prepares the EEG raw data in the testing set 1225 to be used in the trained neural net, e.g., generates spectrograms and snapshots.

The snapshots of the testing set are supplied to the trained neural net, and based on its training, the server 15B (CNN) detects if a seizure is within the snapshot (FIG. 12, 1240). If the neural net detects N consecutive snapshots containing seizure activity, it determines the presence of a seizure. N is a threshold to classify that a seizure is occurring. For example, N may equal 10, such that 10 images must be consecutively classified as containing a seizure prior to determining “seizure” in a patient.

In application, once the model is tested and trained, the CPU in the server 15B executes the model (ML seizure detector 300). For example, as the CPU generates the spectrograms in a manner as described above from a patient 2, the CPU samples the same with a moving window to create the consecutive frames. When N frames are classified as “seizure”, the CPU transmits a signal (such as an alert) to the bedside monitor 200 to generate an alarm. Upon receipt of the signal, the bedside monitor 200 issues the alarm. For example, the bedside monitor 200 may issue an audio alert 310 to alert the bedside clinician 36 to the seizure. In other aspects, the bedside monitor 200 may emit a light as the alarm. In other aspects, the bedside monitor 200 may transmit the alert to an attending physician or nurse's station.

The server 15B may also transmit the generated visualizations, e.g., spectrograms, such as the MPS to the bedside monitor 200. In an aspect of the disclosure, a window may be superposed on the MPS indicating the detected seizure. As noted above, other visualizations, such as one or more relationships of the MPS may be transmitted.

The selection for the abovementioned parameters (sampling window size, type and depth of neural network, threshold of consecutive snapshots) may be empirically determined for example, in a manner described below in example 2.

### Example 1: MPS Seizure Detection by Non-neurophysiologist Physicians After Brief Training

The method of generating spectrograms and displaying the same as illustrated in FIG. 2c and described above was tested for effectiveness in seizure detection and compared with the detection using other methods.

This was a single center, single-blind trial with a convenience sample of neurology residents using the MPS. The primary objective was seizure identification.

Twelve neurology residents (PGY 2-4) were used in the study. Some had experience interpreting traditional EEGs as part of residency training, but none had prior experience using spectrograms for EEG interpretation.

EEGs. EEG records were acquired from the publicly available Children's Hospital Boston and Massachusetts Institute of Technology (CHB-MIT) scalp EEG database as part of PhysioNet.24 The CHB-MIT database include EEGs recorded from 22 children with intractable seizures (5 boys ages 3-22 and 17 girls ages 1.5-19). The database annotation included the start and end time of the seizures. EEGs were selected that were recorded using the international 10-20 system. EEGs were digitized via an ADC having a sampling rate of 256 Hz. Because the focus was seizure detection, rather than seizure counting—EEGs that contained only a single seizure were selected. To mitigate reviewer fatigue, EEGs>4 hours long were excluded. Based these criteria, 101 of 185 available EEGs that contained seizures were selected.

90 of 101 records were randomly selected and allocated into 6 sets of 15 records. In these records, the shortest seizure was 6 s and the longest was 12.5 minutes. For negative controls, 30 records were randomly selected (each 4 hours long, recorded in 10-20 system) from 79 records without seizures, and randomly allocated these 30 records into the 6 sets, 5 records per set. This resulted in 6 sets containing 20 records each, with a 3:1 ratio of seizure to non-seizure records.

The MPS Display. The EEG channels were divided into four groups based upon location in a scalp quadrant in a similar manner as shown in FIG. 10. In each quadrant, the median spectral power was calculated across all channels, per frequency bin per second, creating four median power spectrograms, one for each scalp quadrant. Summing all four spectrograms created a summed median power spectrogram (S-MPS) in a similar manner as shown in FIG. 11b. Taking absolute value difference of the anterior two quadrants and posterior two quadrants produced a paired difference median power spectrogram (D-MPS) in a similar manner as shown in FIG. 11c. Of note, supplementary low-temporal channels were not included, which are sometimes included in the recordings, in the MPS computation.

The frequency power spectrum for each individual channel was calculated with a STFT, which is a moving window calculating a DFT. The window size was 2 s, sliding by 1 s. To optimize frequency resolution, multi-taper spectral estimation was used (DFT size 4096, K=2, WT=2) yielding a frequency resolution of 2 Hz.

Hardware and Software. EEG records were imported into MATLAB v2014b (Mathworks Inc., Natick, Mass.) using the EEGLAB (v13.4.4) software package.25 Signal processing was performed with MATLAB and the CHRONUX (v2.11) software package.22 The user interface and MPS display (S-MPS with D-MPS) were created with MATLAB. In the interface, the viewing window was 15 minutes. Pressing the left or right key scrolled the display 3-minutes backward or forward in time. Using the mouse, the user marked the start and end of a possible seizure. The display was shown on a 23 in. 1920×1080 resolution monitor.

EEG Seizure Review. A board certified pediatric neurologist and neurophysiologist, blinded to the MPS, reviewed the 90 seizure containing EEGs and categorized each seizure into four categories (and sub-categories): generalized (spike-wave, secondarily generalized, or tonic), focal (short [<60 s] and long), low temporal, and ambiguous. Seizures were described as ambiguous if the reviewer felt the raw EEG did not clearly contain a seizure, despite an annotation in the CHB-MIT database.

MPS Seizure Review. A neurologist with qEEG experience reviewed the 90 seizure containing records on the MPS. The reviewer was blinded to both the raw EEGs and the results of the EEG reviewer. Based on visual inspection, the reviewer indicated if the seizure was discernable on the S-MPS, D-MPS, or both.

Trial Design. Each resident first watched a 5-minute video tutorial on seizure recognition using the MPS display. They then learned how to use the computer interface, followed by a post-test containing five spectrograms and an opportunity for feedback.

The video tutorial emphasized three MPS features illustrated in FIG. 8d that distinguished seizures from inter-ictal background: power difference from the background 805, down-sloping resonance bands 800, and power in high frequencies 810. The tutorial further emphasized the importance of down-sloping resonance bands 800, which highlight both the rhythmicity and evolution of a seizure.

The participants were blind to each set's 3:1 seizure to non-seizure composition, but were told each record contained at most one seizure. Using the computer interface, the participant synchronously scrolled through the S-MPS and D-MPS marked individual seizures by recording their start and end time. Detection was considered to be positive if the participant's recorded start and end times overlapped with the database annotation.

Participants were randomized to 1 of 6 sets. Each set was evaluated by two participants. No participant evaluated more than one set. No time limit was imposed, but participants were instructed to ideally spend <1 minute per record.

Results

EEG Seizure Characteristics

EEG Seizure Review. Of the 90 records with seizures, the neurophysiologist's determination was that 27 (30%) were generalized (11 generalized spike-wave, 11 secondarily generalized, and 5 tonic) and 59 (66%) were focal only (29 short, 30 long). Five seizures, all focal, were evident primarily on supplementary low temporal leads. There were 4 (4%) records in the ambiguous category.

MPS Seizure Review. All seizures were visible on the S-MPS, and 31/90 (34%) of seizures were visible on the D-MPS, which is expected because the S-MPS primary intended use is as a generalized seizure visualization, whereas the D-MPS primary intended use is as a high specificity focal seizure visualization.

Overall Detection Characteristics

Sensitivity and Specificity. The mean sensitivity of seizure detection using the MPS across all study participants was 77% (95% confidence interval [CI] 73-88%), and mean specificity was 72% (95% CI 65-83). The mean false negative rate was 0.14 (95% CI 0.09-0.19) per hour, and the false positive rate was 0.07 (95% CI 0.04-0.10) per hour. Longer seizures were more easily identified (r2=0.7; p<0.01). Residents identified 64% (29/45) of seizures <1 minute, compared to 87% (39/45) of seizures >1 min (p<0.05; Chi-square test). The shortest detected seizure was 12 s, and the longest was 12.5 minutes.

Inter-rater Agreement. The mean inter-rater agreement between residents per set was moderate, Cohen's Kappa 0.57 (95% CI 0.51-0.62).

Detection of Seizure Types

Generalized Seizures. For generalized seizures 81% (22/27) (75% [12/16] of primary generalized and 90% [10/11] of secondarily generalized seizures) were detected by at least one resident using the MPS. All four of the primary generalized seizures missed by both residents were tonic seizures. Notably, one of five tonic seizures was detected. Due to the unique EEG features of tonic seizures, they are not detectable on the CDSA. However, likely due to the MPS' specific signal processing, they are faintly visible and was visually identified in one instance.

Focal Seizures. For focal seizures, 86% (51/59) were detected by at least one resident using the MPS. Of the missed focal seizures (n=8), 4 were evident primarily in supplementary low temporal channels. Two seizures were brief (27 and 52 s) and subtle on the raw EEG itself. The remaining two seizures were delta predominant seizures. These were the only delta predominant seizures missed by residents, as they were able to identify 88% (14/16) of delta predominant seizures using the MPS.

The majority of missed focal seizures were those evident primarily on supplementary low temporal channels, which are derived from additional electrodes placed in rare scenarios to evaluate for subtle temporal lobe seizures. Because these electrodes are non-standard, their associated EEG waveforms were not included in the MPS computation, and thus not visible on the MPS.

Visualizing lower power delta predominant seizures is a problem for qEEGs in general, and these seizures are generally not visible on the CDSA. For example, even with five different qEEG modalities and expert review, sensitivity for these seizures ranges 30-42%. However, the MPS detected 88% of these seizures in our data set, suggesting that the MPS may be an improved method for detecting delta predominant seizures.

Ambiguous Cases. Of the 4 ambiguous cases, two cases showed distinct sloped bands and were detected as seizures by both residents on the MPS; the remaining two were not detected by either resident.

S-MPS vs. D-MPS. Visual inspection of the S-MPS found a discernible signal for all seizures, both generalized and focal. On the D-MPS, the reviewer observed signal for 59% (35/59) of focal seizures, 64% (7/11) of secondarily generalized seizures, and none of the primary generalized seizures, which is consistent with the D-MPS design as a high specificity focal seizure visualization; thus, none of the generalized seizures were present and only the distinct focal seizures were visible on the D-MPS. The rationale for the D-MPS was for it to supplement the S-MPS, and its higher specificity was more desirable because while broad spectrum seizure medications work for both focal and generalized seizures, they typically have worse side effect profiles. However, medications for focal seizures have better side effect profiles but have limited effectiveness for generalized seizures. Thus, it is important to clearly identify focal seizures as false positives can lead to treatment of a generalized seizure with a medication for focal seizures, which are often ineffective.

Summary of Findings

This study describes and evaluates a novel median power spectrographic display for seizure detection. Overall, the average sensitivity (77%) and specificity (72%) is at least comparable to previous studies using qEEG techniques. The display of an MPS was effective for focal and generalized seizures.

As described above, the MPS displays high-power, high-frequency discharges as tall and intensely colored. The MPS additionally reveals sloped harmonic bands as a visually salient indicator of rhythmicity. This pattern is particularly helpful for discriminating seizures. In some cases, this may be helpful where there is equipoise on the EEG. For example, two seizures that appeared ambiguous on the raw EEG had sloped bands on the MPS—both were consistently identified by residents.

Spatial Resolution and Seizure Localization. The S-MPS was expected to be a generalized seizure detector, because it sums power across the entire head, and the D-MPS was expected to be a focal seizure detector because it highlights differences between the hemispheres. In practice, however, some seizures can be seen on both the S-MPS and D-MPS. For example, as a focal seizure propagates across the head, the spectral power becomes increasingly prominent in the S-MPS.

Compared to dual-channel hemispheric CDSA, the study demonstrates improved sensitivity (77% [95% CI 73-88%] vs. 70% [95% CI 67-73%]) and comparable specificity (72% [95% CI 65-83%] vs. 68% [95% CI 67-70%]). One possible explanation is that decreasing spatial resolution from quadrants to hemispheres often dilutes lower intensity focal seizures.

It is also possible that additional spectral channels do not improve sensitivity or specificity. Compared to 8-channel CDSA, the MPS display also demonstrates potentially improved sensitivity (77% [95% CI 73-88%] vs. 65% [95% CI 54-75%]) and comparable specificity (72% [95% CI 65-83%] vs. 75% [95% CI 65-84%]). It may be that increasing visual complexity with multi-channel CDSA limits effective interpretation.

The S-MPS, as a single channel, yielded an accuracy of 81% when used by residents in this study. This is promising, as limited data describing accuracy with 5-6 channel CDSA range 69-88%.

Advantageously, because median statistics are robust to outliers, the MPS will be resilient to both noise and artifacts. Formal evaluation with different noise and artifacts will be valuable, as effects of noise and artifacts in qEEGs are understudied.

User Training Time. As described above, training time of a 5-minute tutorial was used which is a ⅔ reduction compared to the shortest previously reported training times. This is likely because of the advantage of using MPS better visualizes seizures with three features: difference from the background, sloped resonance bands, and power in high frequencies for enabling seizure detection, as shown in FIG. 8d.

### Example 2: CNN Training and Performance in Automatic Seizure Detection on the MPS

EEGs from Children's Hospital Boston—Massachusetts Institute of Technology (CHB-MIT) and New York Presbyterian—Weill Cornell Medical Center (NYP-WC) were converted into spectrograms via the MPS method illustrated in FIG. 2c. Images were sampled from spectrograms over seizure and non-seizure locations in a manner that simulated telemetry monitoring (such as illustrated in FIGS. 13a-c). The sampled images were used to train, validate, and test four different CNN models.

EEG Data Set. The CHB-MIT EEGs were acquired from PhysioNet.org. The NYP-WC EEGs were acquired from the NYP-WC clinical EEG database. The mean seizure duration for both data sets was 60 s (6 s-12.5 minutes). CHB-MIT EEGs were collected from 22 patients (ages 1.5-19). The waveforms were digitized at a sampling rate of 256 Hz. The EEGs included annotations of the seizure's start and end times identified by neurophysiologists at CHB. Annotations were further verified by a neurophysiologist at Weill Cornell. There were 130 EEGs with 177 seizures, and 549 EEGs without seizures in this data set.

NYP-WC EEGs were collected from a convenience sample of 12 patients (ages 18-99). The waveforms were digitized at a sampling rate of 256 Hz. The EEGs included seizure's start and end times identified by the neurophysiologist at the time of care. These annotations were further independently verified by two neurophysiologists. There were 12 EEGs containing 33 seizures. All EEGs contained at least one seizure.

Spectrogram Snapshot Images. EEGs waveforms (channels) were converted to MPS using the method described above in FIG. 2c, with a single spectrogram representing EEG channels from all four scalp quadrants similar to the S-MPS spectrogram illustrated in FIG. 11b (but in gray-scale). Snapshot images (also referred to as snapshots or frames) of gray-scale S-MPS were obtain in a manner as illustrated in FIGS. 13a-c, depending on whether the snapshot made a seizure in the image. For each snapshot image, the pixel height represented a frequency bin of 0.125 Hz (Fs [sampling rate]=256, N [length of Fast Fourier transform]=2048, Fs/N=0.125 Hz). Thus, the total image height (160 pixels) included the 0-20 Hz frequency bands. The snapshots were obtained with a 120 s sliding window, advancing at is increments. Thus, pixel width represented is of elapsed time, and all snapshot images were 160×120 pixels.

Snapshots of spectrogram images containing seizures were obtained starting with the seizure's leading edge in the middle of the 120 s window. As the seizure's leading edge traveled across the sliding window at is increments, a snapshot of the window was taken, resulting in 60 snapshot images per seizure. This method was used to simulate telemetry monitoring where a 120 s wide detection window would travel across the spectrogram. Furthermore, a 120 s detection window was selected to coincide with the maximum clinically recommended duration to initiate treatment on a patient with continuous or near continuous seizures. Note that for seizures >120 s in duration, only the first 120 s were sampled.

Snapshots of spectrogram images without seizures were obtained as above, but with two different methods in selecting the starting locations between the CHB-MIT and NYP-WC spectrograms. For CHB-MIT spectrograms, a random start location was set on each of 177 randomly selected spectrograms without seizures, and 60 snapshot images obtained from each location. For NYP-WC spectrograms, because all spectrograms contained at least one seizure, seizure-free snapshot images were obtained from spectrogram epochs before the first seizure and after the last seizure. The snapshot locations were randomly selected from epochs where the sliding window would not overlap with any seizures.

Spectrogram Review. All spectrograms in this study were also reviewed for seizure visibility. From previous work, all 177 CHB-MIT seizures were visible on the MPS (MPS calculated from four scalp quadrants and then summed). For the NYP-WC EEGs, all MPS underwent blinded review by a neurophysiologist. 17/33 seizures were not visually discernable on the spectrogram, with 8 of them from one patient (this patient had very subtle seizures on EEG, which without corresponding video recording of the patient that correlating the seizure's physical manifestation to the EEG, the seizure would have otherwise been not likely identified on visual analysis of the EEG waveform alone or with any qEEG visualization).

Spectrogram Image Partitioning. 90% of the CHB-MIT images (snapshots) were partitioned for training and cross-validation of the CNNs. The other 10% was set aside for testing. All NYP-WC (snapshots) images were used for testing only. Snapshot Images were partitioned based on seizures (i.e. images belonging to an individual seizure were partitioned together into one group). Because not all seizures were visible on the NYP-WC images, a subset containing only spectrogram-visible seizures was also created, which better represents what the bedside clinician would observe (i.e., only seizures that are visible on the spectrogram).

CNN Architecture. As described above, the CNN is a specific type of deep learning neural network model that is composed of nested layers of convolutions and sub-sampling. There are many CNN architectures that differ depending on the composition and connections among different layers. In this study, a VGG-net was used. The VGG-net is a well-known CNN architecture. The VGG-net was selected because of its modular block design and high performance in the ImageNet classification task. There were four CNN models (FIG. 15b, Net 1, Net 2, Net 3 and Net 4) used in this study. All are based on VGG-net, and each containing a different combination of convolution layer sets connected in series to classification layers C. Each CNN model consisted of a one or more sets (1, 2, 3, or 4) connected to a group of layers functioning as the final classifier C as shown in FIGS. 15a and 15b. The CNN models differed based on the number of consecutive convolution layer set prior to the layers involved in classification. For example, the fourth model Net 4 consisted of the most number of convolution layer sets prior to classification (FIG. 15a, 15b, Net 4 had layer sets 1-4), with the other three models containing fewer convolution layers (FIG. 15b). As shown in FIG. 15b, Net 1 had layer set 1; Net 2 had layer sets 1 and 2; Net 3 has layer sets 1, 2 and 3. The output of each set or group of convolution layers is a ‘higher-level’ feature representation of the input image (snapshot). This new representation of the input image (snapshot) can then be further processed by additional sets or groups of convolution layers, until the final ‘optimal’ image representation is learned in the classification layers C. This process is remarkably similar to the image processing in the human visual cortex.

CNN Training/Validation. The CNN's convolution layers can be conceptualized as a series of weighted functions that extract the ‘optimal’ features describing a spectrographic seizure. The training process is to determine the optimal weights for these functions to achieve this goal. All CNNs were trained using stochastic gradient descent with momentum (batch size=256, γ=0.9, learning rate=0.01). To alleviate overfitting, L2 regularization (with regularization parameter 0.0005) was used and 0.5 dropout rate was used in the fully connected (FC) layers. All training proceeded for 50 epochs in all CNNs, at each cross-validation step. After cross-validation, the CNN was trained on all samples from the training/validation set. All CNNs were trained with MATLAB 2017a (Mathworks, Natick, Mass.) using a Titan Xp GPU (NVIDIA, Santa Clara, Calif.).

CNN Testing. The trained CNN was used to detect seizures from three test sets: the CHB-MIT test set, the NYP-WC test set with all spectrograms, and the NYP-WC test set with those spectrograms with visible seizures only. Detection performance was evaluated at the level of the seizures. The presence of a seizure was determined by the N number of consecutive snapshot images classified as containing a seizure (e.g., for N=10, 10 snapshot images must be consecutively classified as containing a seizure before calling a positive seizure detection). N was varied 1-60, with sensitivity and specificity calculated at each N.

Results

As shown in FIG. 16b, Nets 1-3 had average validation accuracy above 85% after a certain number of iterations. The shading (blue ribbon indicates 95% confidence interval (CI). The number of iterations to reach a certain percentage of validation accuracy was different for Net 1, Net 2 and Net 3. As shown in FIG. 16b, Nets 1-3 had average sensitivity and specificity between 80-90%, though the 95% confidence interval was wider for Net 3 (FIG. 16b). In FIG. 16b, average sensitivity is shown in blue and average specificity is shown in red.

For the CHB-MIT test set, for Net 1-Net 3, identification of a seizure positive event varied based on the number of N consecutively detected seizure positive images. For Net 1-Net 3, there was a range of N where seizure detection was sensitivity and specificity >90%. The range is shown in shading in FIG. 17. Sensitivity is shown in red and specificity is shown in blue. The different models, Net 1, Net 2 and Net had a different range where sensitivity and specificity was >90%. As seen in FIG. 17, this range was wider for the medium complexity CNNs such as Net 2 (N=11-56) and Net 3 (N=13-35) compared with the least complex such as Net 1 (N=3-10).

For the NYP-WC test set, in the subset of spectrograms with visible seizures, Net 2 and 3 had ranges of N consecutive seizure-positive images that had a sensitivity >90% and specificity >75%. The range is shown in shading. The ranges of N were much narrower compared to their counter-part CHB-MIT results (Net 2: 8-10 vs. 11-56 and Net 3: 5-8 vs. 13-35) as shown in FIG. 18. Furthermore, while sensitivity for Nets 2 and 3 remained >90%, there was a decrease in specificity to 75-80% when compared to their prior CHB-MIT performance (FIG. 17 v. FIG. 18). Sensitivity is shown in red and specificity is shown in blue.

CNN Performance. While trained using CHB-MIT spectrograms from primarily pediatric patients, the CNN models in this study achieved >90% sensitivity and 75-80% specificity seizure detection on adult NYP-WC spectrograms, which suggest both reasonable model performance and more importantly, potential generalizability to many clinical EEGs.

While Nets 1-3 converged during training, Net 4 did not. This is likely related to image complexity of the spectrographic seizures. Because the sloped banding pattern characteristic of spectrographic seizures consists of medium level image features (combinations of edges, corners, and shading), medium complexity CNNs may be better suited to recognize these features. In CNNs, each subsequent convolution sets of layers extracts a higher-level summary of the features from the previous set of layers, and thus some details from the previous lower-level features may be lost. This is advantageous in increasing the CNN's generalizability (i.e. recognizing higher-level image features). However, after too many convolution sets of layers, the necessary detail required to achieve the classification of medium level images features may be lost. The CNN does not converge on a solution, which is likely the case in Net 4, the more complex of the four CNN models.

While Net 4 did not converge in this study, this does not mean that Net 4 will never converge when different and/or more training images are used.

While performance on the CHB-MIT test set was comparable to the cross-validation results, performance on the NYP-WC test set was less specific with a narrower range of N for optimal seizure detection. This suggests some overfitting because all CNN models were trained on the CHB-MIT data. However, overfitting is a common problem in neural nets, and it persisted in the CNNs despite using dropout and L2 regularization to mitigate its effects. There are three likely reasons for overfitting in this study. First, the seizures in our training set originate from a small patient cohort and the number of seizures is unevenly distributed across patients. While the sloped banding pattern on the spectrogram is prototypic of seizures, most patients have specific versions of these patterns. Thus, CNNs trained on CHB-MIT spectrograms, while able to generally recognize the sloped bands, are more predisposed to recognize the types of banding patterns in the CHB-MIT spectrograms. Second, the CHB-MIT training spectrograms and NYP-WC test spectrograms are from two different institutions. Minor differences in EEG acquisition (e.g. variations in electrodes, electrode gels, and other acquisition techniques) between institutions may introduce small but systematic differences between CHB-MIT and NYP-WC spectrograms. Third, the CHB-MIT and NYP-WC patients have different demographics (children ages 1.5-19 vs. adults ages 18-99). Children and adults have different prevailing etiologies for their seizures, which can systematically affect the seizure's electrographic profile. This can lead to more variation in spectrographic morphology between CHB-MIT and NYP-WC seizures, which then leads decreased performance when a CHB-MIT trained model is tested on the NYP-WC data. These above consideration will provide guidance in the selection of the training images for fine tuning of the model.

The CNN's real-world performance will be influenced by the patient population's underlying seizure prevalence. In critically ill patients, the prevalence ranges 8-50%, and assuming the CNN's lower end performance (90% sensitivity, 75% specificity), this translates to a positive predictive value (PPV) of 25-78% and negative predictive value (NPV) of 88-98%. The high NPV indicates once again that the CNN is better used for seizure screening. Additionally, the wide PPV range underscores the clinician's role in judiciously selecting patients for cEEG, as those patients with higher seizure likelihood will derive more benefit from cEEG monitoring in general, and CNN seizure auto-detection will be more accurate.

Aspects of the disclosure address one or more deficiencies in EEG systems. For example, automated spectrographic seizure detection as described herein can help address certain issues by either providing telemetry seizure monitoring for the bedside clinician or augmenting seizure screening for the neurophysiologist.

For the bedside physician, the MPS offers a concise EEG visualization where seizures are easily recognizable. The application of the automated detection described herein provides automated telemetry monitoring for seizures may also provide quicker intervention 40 especially where it is not feasible for a clinician to constantly monitor the bedside monitor 200. The automated detection is achieved using machine learning trained on sampled spectrographic images to simulate a how a clinician would monitor the MPS frame by frame (i.e. telemetry monitoring). For example, in example 2, spectrogram images were sampled from a 120 s moving window. Within this 120 s window, images containing a seizure were labelled when the seizure first reached the middle of the window. In practice, this means that the automatic detection may not detect a seizure until 60 s after it had initially occurred. This is clinically acceptable as most seizures self-remit between 30-60 s, and it is recognizing that a seizure has occurred and initiating treatment within a reasonable time (on the order of minutes to tens of minutes) that lead to improved outcomes. Furthermore, the automated seizure detection performance in example 2 (>90% sensitivity and 75-80% specificity) is comparable to clinician (non-neurophysiologist) performance in detecting seizures on the MPS (73-88% sensitivity and 65-83% specificity) in example 1. Thus, indicating that the machine learning based seizure auto-detection is comparable to a clinician constantly monitoring the MPS. Additionally, having the MPS displayed at the bedside allows for the clinician to verify the automated seizure detection after the clinician has been alerted.

For the neurophysiologist, the automated detection is particularly helpful during their review of long (>24 hr) EEG records. Further, the automated detection can augment existing workflow by detecting potential seizures and highlighting them as areas of interest for the neurophysiologist. This may increase review speed, which would alleviate the increasing demand for more cEEG monitoring.

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided, e.g., a computer program product.

The computer readable medium could be a computer readable storage device or a computer readable signal medium. A computer readable storage device, may be, for example, a magnetic, optical, electronic, electromagnetic, infrared, or semiconductor system, apparatus, or device, or any suitable combination of the foregoing; however, the computer readable storage device is not limited to these examples except a computer readable storage device excludes computer readable signal medium. Additional examples of the computer readable storage device can include: a portable computer diskette, a hard disk, a magnetic storage device, a portable compact disc read-only memory (CD-ROM), a random access memory (RAM), a read-only memory (ROM), an erasable programmable read-only memory (EPROM or Flash memory), an optical storage device, or any appropriate combination of the foregoing; however, the computer readable storage device is also not limited to these examples. Any tangible medium that can contain, or store, a program for use by or in connection with an instruction execution system, apparatus, or device could be a computer readable storage device.

A computer readable signal medium may include a propagated data signal with computer readable program code embodied therein, such as, but not limited to, in baseband or as part of a carrier wave. A propagated signal may take any of a plurality of forms, including, but not limited to, electro-magnetic, optical, or any suitable combination thereof. A computer readable signal medium may be any computer readable medium (exclusive of computer readable storage device) that can communicate, propagate, or transport a program for use by or in connection with a system, apparatus, or device. Program code embodied on a computer readable signal medium may be transmitted using any appropriate medium, including but not limited to wireless, wired, optical fiber cable, RF, etc., or any suitable combination of the foregoing.

The terms “Processor”, as may be used in the present disclosure may include a variety of combinations of fixed and/or portable computer hardware, software, peripherals, and storage devices. The “Processor” may include a plurality of individual components that are networked or otherwise linked to perform collaboratively, or may include one or more stand-alone components. The hardware and software components of the “Processor” of the present disclosure may include and may be included within fixed and portable devices such as desktop, laptop, and/or server, and network of servers (cloud).

The terminology used herein is for the purpose of describing particular embodiments only and is not intended to be limiting the scope of the disclosure and is not intended to be exhaustive. Many modifications and variations will be apparent to those of ordinary skill in the art without departing from the scope and spirit of the disclosure.

What is claimed is:

1. A method comprising:
obtaining electroencephalogram (EEG) waveforms from a plurality of EEG channels, where a channel comprises any pair-wise combination of EEG electrodes, respectively, where the electrodes are in contact with a scalp of a subject;
converting the received EEG waveform into a spectrogram, showing EEG spectral power as a function of frequency and time, for each EEG waveform;
grouping spectrograms corresponding to channels into a plurality of groups, wherein at least two spectrograms are in each group;
for each group, aggregating the spectrograms via a median power spectrogram (MPS);
calculating one or more relationships between the MPS from at least two groups; and
displaying the one or more relationships on a bedside monitor.

2. The method of claim 1, wherein one of the relationships is calculated by summing the MPS from at least two groups.

3. The method of claim 1 or claim 2, wherein one of the relationships is calculated by taking a difference between the MPS from at least two groups.

4. The method of claims 1 to 3, wherein there are at least four groups, and wherein two of the relationships are calculated by respectively summing the MPS from at least two groups.

5. The method of claims 1 to 4, wherein the one or more relationships are separately displayed on the bedside monitor.

6. The method of claims 1 to 5, wherein the grouping is based on location of the electrodes on the scalp.

7. The method of claim 6, wherein there are four groups, the four groups include anterior left and anterior right, posterior left and posterior right.

8. The method of claim 7, wherein the MPS for the anterior left and the anterior right are summed.

9. The method of claim 7 or claim 8, wherein the MPS for the posterior left and the posterior right are summed.

10. The method of claims 7 to 9, wherein a difference between the MPS for the anterior left and the anterior right is calculated.

11. The method of claims 7 to 10, wherein a difference between the MPS for the posterior left and the posterior right is calculated.

12. The method of claim 11, wherein the size and color of lines is based on intensity and frequency.

13. The method of claim 12, wherein rhythmicity and intensity are conveyed.

14. The method of claim 13, wherein sloped harmonic bands indicate evolving rhythmicity.

15. The method of claim 1, wherein the obtained EEG waveforms are scaled using the multi-taper spectral estimation method.

16. The method of claim 15, wherein the converting of the scaled EEG waveforms into the spectrogram is based on a short time Fourier transform (STFT)

17. The method of claims 1 to 11 and 16, further comprising automatically detecting a presence of a seizure.

18. The method of claim 17, further comprising generating an alert when a seizure is automatically detected and transmitting the alert.

19. The method of claim 18, further comprising, in response to receiving the alert, displaying the alert on the bedside monitor, generating a sound or transmitting the alert, by the bedside monitor in response to receiving the alert.

20. A method comprising:
obtaining electroencephalogram (EEG) waveforms from a plurality of EEG channels, where a channel comprises any pair-wise combination of EEG electrodes, respectively, where the electrodes are in contact with a scalp of a subject;
converting the obtained EEG waveform into a spectrogram, showing EEG spectral power as a function of frequency and time, for each EEG waveform;
grouping spectrograms corresponding to channels into a group aggregating the spectrograms into a median power spectrogram (MPS) for the group; and
determining whether the subject has a seizure using a model created from a plurality of snapshot images of spectrograms from a plurality of patients and the MPS.

21. The method of claim 20, further comprising generating the model.

22. The method of claim 21, wherein the generating of the model comprising:
obtaining a plurality of snapshot images of known seizures and a plurality of snapshot images of known non-seizures;
dividing the plurality of snapshot images of known seizures and the plurality of snapshot images of known non-seizures into a training set of snapshot images and a testing set of snapshot images;
for the training set of snapshot images, classifying each snapshot image by applying an artificial neural network to train the model; and
testing the artificial neural network using the testing set of snapshot images.

23. The method of claim 22, wherein the artificial neural network comprises a plurality of layers, the plurality of layers including a plurality of layer sets, each layer set having a different convolution operation.

24. The method of claim 23, wherein each layer set is a convolution operation having X by X pixel convolution filters, where X is the pixel size and is applied at Y-pixel steps, where Y is the step size.

25. The method of claim 24, wherein the number of X by X pixel convolution filters is different for each layer set.

26. The method of claim 21, further comprising:
calculating an MPS for a plurality of groups; and
calculating a relationship between the MPS from at least two groups.

27. The method of claim 26, wherein the determining includes obtaining snapshot images from the MPS aggregated or snapshot images from the relationship between the MPS using a moving window.

28. The method of claim 27, wherein the subject is determined to have a seizure when a threshold number of consecutive snapshot images are classified as a seizure.

29. The method claim 28, wherein the threshold number is 10.

30. The method of claim 2, wherein snapshot images are obtained by a moving window with a set movement step.

31. The method of claim 22, wherein the obtaining comprises receiving historical EEG raw data from a database from a plurality of patients, the historical EEG raw data including EEG raw data from a plurality of patient determined to have a seizure and a EEG raw data from a plurality of patients determined not to have a seizure and generating a MPS from the historical EEG raw data for each patient, and for each MPS, generating snapshot images of the MPS using a moving window to generate a plurality of snapshots, and classifying each snapshot as a seizure image and non-seizure image.

32. The method of claims 20 to 31, receiving a request from a client terminal to review the electroencephalogram (EEG) waveforms and/or the MPS and in response to the request, transmitting the EEG waveforms and/or the MPS to the client terminal.

33. A server comprising:
a network interface;
a storage configured to store digitized electroencephalogram (EEG) signals received via the network interface, the EEG signals were obtained from electrodes in contact with a scalp of a subject; and
a processor configured to:
retrieve the EEG signals from the storage;
group EEG signals into a plurality of EEG channels, where a channel comprises any pair-wise combination of EEG signals, respectively;
convert the pair-wise combination of EEG signals of the channel into a spectrogram, showing EEG spectral power as a function of frequency and time, for each channel;
group spectrograms corresponding to channels into a plurality of groups, wherein at least two spectrograms are in each group;
for each group, aggregate the spectrograms via a median power spectrogram (MPS);
calculate one or more relationships between the MPS from at least two groups; and
transmit the MPS or the one or more relationships between the MPS from at least two groups to a bedside monitor.

34. The server of claim 33, wherein the processor is further configured to automatically detect a seizure in a patient by analyzing the MPS or a relationship between the MPS from at least two groups.

35. The server of claim 34, wherein the processor is further configured to transmit an alert when a seizure is automatically detected.

36. The server of claims 33-35, wherein the processor is further configured to store the MPS or the one or more relationships between the MPS from at least two groups in the storage.

37. The server of claim 36, wherein the processor is configured to receive via the network interface a request from a client terminal to view of the MPS and/or the one or more relationships between the MPS from at least two groups in the storage and in response to the receipt of the request, cause the transmission of the MPS and/or the one or more relationships between the MPS from at least two groups to the client terminal via the network interface.

38. A server comprising:
a network interface;
a storage configured to store digitized electroencephalogram (EEG) signals received via the network interface, the EEG signals were obtained from electrodes in contact with a scalp of a subject; and
a processor configured to:
retrieve the EEG signals from the storage;
group EEG signals into a plurality of EEG channels, where a channel comprises any pair-wise combination of EEG signals, respectively;
convert the pair-wise combination of EEG signals of the channel into a spectrogram, showing EEG spectral power as a function of frequency and time, for each channel;
group spectrograms corresponding to channels into a group
aggregate the spectrograms via a median power spectrogram (MPS) for the group; and
determine whether the subject has a seizure using a model creates from a plurality of snapshot images of spectrograms from a plurality of patients and the MPS.
